(19) United States
(12) Patent Application Publication
(10) Pub. No.: US 2023/0248734 A1
(43) Pub. Date: Aug. 10, 2023

(54) ANTIVIRAL COMPOSITION OBTAINED THROUGH DRUG REPOSITIONING

(71) Applicant: KOREA RESEARCH INSTITUTE OF CHEMICAL TECHNOLOGY, Daejeon (KR)

(72) Inventors: Soo Bong HAN, Daejeon (KR); Jin Soo SHIN, Daejeon (KR); Mee Hyein KIM, Daejeon (KR); Eunhye JUNG, Daejeon (KR); Ye Jin JANG, Daejeon (KR); Hyuk LEE, Daejeon (KR); Myoungkyu LEE, Daejeon (KR); Dongsu KIM, Daejeon (KR); Chang Soo YUN, Daejeon (KR); Young Sik JUNG, Daejeon (KR)

(73) Assignee: KOREA RESEARCH INSTITUTE OF CHEMICAL TECHNOLOGY, Daejeon (KR)

(21) Appl. No.: 18/001,333

(22) PCT Filed: Jun. 8, 2021
(86) PCT No.: PCT/KR2021/007104

§ 371 Date: Dec. 9, 2022

(30) Foreign Application Priority Data
Jun. 9, 2020 (KR) 10-2020-0069697

(51) Int. Cl. A61K 31/53; A61K 31/495; A61K 31/138; A61K 31/4402; A61K 31/137; A61K 31/4164; A61K 31/451; A61K 31/452; A61K 31/4453; A61K 31/40; A61K 31/445; A61P 31/14; G01N 33/50

(52) U.S. Cl. CPC A61K 31/53; A61K 31/495; A61K 31/138; A61K 31/4402; A61K 31/137; A61K 31/4164; A61K 31/451; A61K 31/452; A61K 31/4453; A61K 31/40; A61K 31/445; A61P 31/14; G01N 33/5091

(57) ABSTRACT

The present invention relates to the development of a composition for the treatment of novel SARS-coronavirus-2 infection. The drug efficacy was evaluated through CPE (Cytopathic Effect)-based assay method that measures how long the cells survive by infecting SARS-CoV-2 after receiving a drug library previously approved as a treatment for a specific disease from the Korea Chemical Bank and treating Vero cells with the drug, and an image-based assay that measures how much virus is reduced in infected cells. Through this, 16 kinds of drugs effective in the treatment of SARS-CoV-2 were re-positioned.

### BACKGROUND

### Technical Field

The present invention relates to an antiviral composition by drug repositioning.

### Background Art

On Dec. 31, 2019, a case of respiratory pneumonia of unknown cause was first reported to the WHO in Wuhan, Hubei Province, China. Since then, through human-to-human transmission, it has rapidly spread beyond the community's infection to the world. It has been found that this virus is one of the coronaviruses and is a novel virus that is genetically different from the previously known SARS coronaviruses. As of Feb. 13, 2020, as reported there were more than 60,000 confirmed cases and the fatality rate was 2.2%. By Mar. 11, 2020, WHO declared the 2019-type coronavirus infection (COVID-19) a global pandemic, which is prevalent in more than 100 countries, including Korea.

It is known that Coronavirus is a positive single-stranded (+ssRNA) virus with a characteristic that the Spike (S) protein in the envelope shows a crown shape on an electron microscope, and is a round or oval virus with a size of 60-140 nm. The subgroup of coronavirides is Orthocoronavirinae, which is classified into four genera: alphaCoV, betaCoV, deltaCoV, and gammaCov. Among them, only alphaCoV and betaCoV infect mammals, while deltaCoV and gammaCov infect birds and some mammals. So far, seven types of coronaviruses that can infect humans are known. Among them, SARS-CoV and MERS-CoV belonging to betaCoV cause severe respiratory infectious diseases characterized by acute respiratory symptoms. Mortality rates associated with SARS-CoV and MERS-CoV are up to 10% and 35%, respectively.

The genomic sequence isolated from a patient with atypical pneumonia in Wuhan in the early stages of COVID-19 was analyzed to have 89% nucleotide homology with bat SARS-like-CoVZXC21 and 82% homology with SARS-CoV, and was found to belong to the same betaCoV, and was named SARS-CoV-2 (SARS-CoV2). Recently, research to develop a treatment for COVID-19 is rapidly increasing, but no drugs have been approved as a treatment so far.

Novel SARS-coronavirus-2 is a virus with a single-stranded positive RNA genome of about 30,000 base pairs. A virus whose surface is surrounded by an envelope. Common Covid-19 patients show relatively mild symptoms such as elevated body temperature, cough, and chest pain, but some patients develop serious diseases such as dyspnea and pneumonia, leading to death. To date, no approved therapeutics or vaccines have been reported, and patients are being treated with symptomatic therapy that relieves symptoms. Therefore, there is an urgent need to develop therapeutic drugs. Drug repositioning is a method of developing new drugs with different indications using FDA-approved drugs with high safety and clinical applicability. It has the advantage of rapidly developing drugs using well-known pharmacological properties and drugs that have undergone many clinical trials. Drugs that inhibit the novel SARS-coronavirus-2 infection can be screened using FDA-approved drugs, and through this, it will be possible to rapidly develop a therapeutic agent.

### BRIEF DESCRIPTION OF THE INVENTION

### Technical Problem

The present invention has been derived from the above needs, and the present inventors completed the present invention by confirming the effect on SARS-CoV2 by exporting a drug library previously approved as a treatment for a specific disease from the Korea Compound Bank.

### Technical Solution

In order to solve the above problems, the present invention provides an antiviral composition comprising one or more compounds selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

Also, the present invention provides a pharmaceutical composition for preventing or treating a viral infection comprising one or more compounds selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

As another example, the present invention provides a food composition for preventing or improving viral infection comprising at least one compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

As another example, the present invention provides a method for screening SARS-coronavirus-2 inhibitory drug, comprising:

A step of administering the drug to be screened to Vero cells;

A step of infecting SARS-coronavirus-2;

A step of measuring cell death by a microscope or MTS method;

A step of reacting with an antibody specific for double-stranded RNA; and

A step of treating one or more compounds selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden as a control.

### Effects of the Invention

The drugs selected according to the present invention exhibit excellent antiviral effects against novel SARS-coronavirus-2 and have high safety and clinical applicability, and thus can be usefully used to develop therapeutic agents for novel SARS-coronavirus-2 infection.

### DETAILED DESCRIPTION OF THE INVENTION

Hereinafter, preferred embodiments of the present invention will be described in detail. In addition, in the following description, many specific details such as specific components are shown, which are provided to help a more general understanding of the present invention, and it will be apparent to those skilled in the art that the present invention may be practiced without these specific details. And, in describing the present invention, if it is determined that a detailed description of a related known function or configuration may unnecessarily obscure the gist of the present invention, the detailed description thereof will be omitted.

As used herein, the term “prevention” refers to inhibiting or delaying the occurrence of a disease from the cause.

As used herein, the term “treatment” means to stop the progression of damage by suppressing the progression and/or exacerbation of symptoms even if not completely cured, or to ameliorate some or all of the symptoms to guide them in the direction of healing.

In order to achieve the object of the present invention, the present invention provides an antiviral composition comprising at least one compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

In order to achieve the object of the present invention, the present invention provides a pharmaceutical composition for preventing or treating viral infection comprising at least one compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

Almitrine bismesylate (trade name: Duxil) according to an embodiment of the present invention is a diphenylmethylpyrrazine derivative and is classified as a respiratory stimulating factor in ATC. It is used as a treatment for chronic obstructive pulmonary disease by acting as an agonist of peripheral chemoreceptors and improving respiration.

Hydroxyzine dihydrochloride (trade names Di-Paralene, Mantadil, Pruresidine, Trihistan) is an antihistamine sold under the brand name Atarax. It is used as a treatment for motion sickness, itching, anxiety and nausea.

Chlorcyclizine hydrochloride is a first-generation antihistamine of the diphenylmethylpiperazine group. It is primarily used to treat allergic symptoms such as rhinitis, hives and itching, and can also be used as an emetic agent. In addition to antihistamine effects, it also has anticholinergic, antisteroidal and local anesthetic properties.

Cinnarizine is an antihistamine and calcium channel inhibitor of the diphenylmethylpiperazine group. It is known to promote cerebral blood flow, and is used to treat brain tumors, post-traumatic brain symptoms, and cerebral arteriosclerosis. However, it is most commonly used as a treatment for nausea and vomiting caused by motion sickness or as a treatment for chemotherapy, dizziness, or Meniere's disease.

Cyclizine, sold under several brands, is a drug used to treat and prevent nausea, vomiting, and vertigo caused by motion sickness or vertigo. It can also be used for nausea after general anesthesia or nausea developed with opioid use.

Orphenadrine citrate is an ethanolamine antihistamine anticholinergic drug sold under many brand names worldwide. Closely related to diphenhydramine. It is used to treat muscle pain and to help control movement in Parkinson's disease, but has been replaced by a new drug.

Brompheniramine maleate is an antihistamine sold under the brand name Dimetapp. It is used to treat symptoms of colds and allergic rhinitis such as runny nose, itchy eyes, watery eyes, and sneezing. It is a first-generation antihistamine and one of the drugs with the highest anticholinergic activity.

Tolterodine tartrate is a drug sold under the brand Detrol used to treat frequent urination, or incontinence.

Cibenzoline succinate is an antiarrhythmic drug.

Zimelidine dihydrochloride monohydrate (trade names Zimeldine, Normud, Zelmid) was one of the first selective serotonin reuptake inhibitor antidepressants on the market. It is structurally different from other antidepressants as pyridylallylamine.

Diphenoxylate hydrochloride is a centrally active opioid drug in the phenylpiperidine class used together with atropine for the treatment of diarrhea. Used to slow intestinal contractions.

Pipenzolate bromide is an antimuscarinic drug. It binds to the muscarinic acetylcholine receptor and acts as an antagonist to inhibit acetylcholine.

Pridinol methansulfonate is an antiparkinsonian and anticholinergic drug used as a muscle relaxant.

Procyclidine hydrochloride is an anticholinergic drug, used in the treatment of drug-induced Parkinson's disease, a psychosis, and acute dystonia, and is also used in idiopathic or secondary myotonia.

Fexofenadine hydrochloride, sold under the trade names Allegra & FX 24, is a second-generation antihistamine used to treat allergic conditions such as hay fever and urticaria.

Biperiden is a drug used to treat Parkinson's disease and certain drug-induced movement disorders sold under the brand name Akineton.

Also, in the pharmaceutical composition according to an embodiment of the present invention, the virus is selected from coronavirus, and preferably is SARS-coronavirus-2, SARS or MERS, but is not limited thereto.

Also, in the pharmaceutical composition according to an embodiment of the present invention, the viral infection may include flu, cold, sore throat, bronchitis, or pneumonia, but is not limited thereto as long as it is a disease caused by a viral infection.

Also, in the pharmaceutical composition according to an embodiment of the present invention, the composition may include an additional component effective against viral infection, and the additional component may include any component known to be effective against viral infection, including compounds and natural products.

The composition of the present invention may further include suitable carriers, excipients and diluents commonly used in the preparation of the composition.

The pharmaceutical composition of the present invention may be used alone or in combination with methods using surgery, drug treatment, and biological response modifiers for the prevention and treatment of diseases.

The pharmaceutical composition of the present invention may be formulated in the form of oral dosage forms such as powders, granules, tablets, capsules, suspensions, emulsions, syrups, aerosols, external preparations, suppositories, and sterile injection solutions according to conventional methods, and is more preferably powers, tablets, capsules, injections, and a liquid.

Such formulation can be carried out by a method commonly performed in the pharmaceutical field, and preferably formulated according to each disease or component using the method disclosed in Remington's Pharmaceutical Science, Mack Publishing Company, Easton Pa.

Carriers, excipients and diluents that may be included in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil, and the like.

In the case of formulation, it may be prepared by additionally using diluents or excipients such as commonly used fillers, extenders, binders, wetting agents, disintegrants, and surfactants.

Solid preparations for oral administration include tablets, pills, powders, granules, capsules, etc., and such solid preparations include at least one excipient, for example, starch, calcium carbonate, sucrose or lactose, gelatin, etc. In addition to simple excipients, lubricants such as magnesium stearate and talc are also used.

Liquid formulations for oral administration include suspensions, solutions, emulsions, syrups, etc., and various excipients such as wetting agents, sweeteners, fragrances, and preservatives may be included in addition to commonly used simple diluents such as water and liquid paraffin. Formulations for parenteral administration include sterile aqueous solutions, non-aqueous solutions, suspensions, emulsions, freeze-dried preparations, and suppositories. As the non-aqueous agent and suspending agent, propylene glycol, polyethylene glycol, vegetable oils such as olive oil, and injectable esters such as ethyl oleate may be used. As the base of the suppository, witepsol, macrogol, tween, cacao butter, laurin, glycerogelatin, etc. can be used.

The preferred dosage of the composition of the present invention varies depending on the condition and weight of the patient, the degree of disease, the drug form, the route of administration, and the duration, but may be appropriately selected by those skilled in the art. However, for a desirable effect, the composition of the present invention may be included in an amount of 0.01 to 99.9% by weight, preferably 0.1 to 99% by weight per day. The daily dose may be about 0.1 to 1,000 mg/kg, preferably 100 to 300 mg/kg.

Additionally, in order to achieve the object of the present invention, the present invention provides a food composition for preventing or improving viral infection comprising at least one compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

The food composition includes, for example, various general foods, beverages, gum, tea, vitamin complexes, and the like.

Also, the component may be added to food or beverage for the purpose of preventing disease. In this case, the amount of the component in the food or beverage may be added in 0.01 to 15% by weight of the total food weight, and the health drink composition may be added in a ratio of 0.02 to 5 g, preferably 0.3 to 1 g based on 100 g.

The health functional beverage composition of the present invention is not particularly limited in other components other than containing the above components as an essential component in the indicated ratio, and may contain additional components such as various flavoring agents or natural carbohydrates like a conventional beverage. Examples of the natural carbohydrates include monosaccharides such as glucose, fructose, etc.; disaccharides such as maltose, sucrose and the like; and polysaccharides, for example, conventional sugars such as dextrin, cyclodextrin, and the like, and sugar alcohols such as xylitol, sorbitol, and erythritol. As a flavoring agent other than those described above, as natural flavoring agent, taumatin, stevia extract, such as rebaudioside A, glycyrrhizin, etc.; and synthetic flavoring agents such as saccharin, aspartame and the like may be advantageously used. The proportion of the natural carbohydrate is generally about 1 to 20 g, preferably about 5 to 12 g per 100 g of the composition of the present invention.

In addition to the above, the ingredients of the present invention may contain various nutrients, vitamins, minerals (electrolytes), synthetic and natural flavoring agents, colorants and thickeners (cheese, chocolate, etc.), pectic acid and its salts, alginic acid and its salts, organic acids, protective colloidal thickeners, pH regulators, stability topical, preservatives, glycerin, alcohol, a carbonation agent used in carbonated beverages, and the like. In addition, the component of the present invention may contain the pulp for the production of natural fruit juice, fruit juice beverage and vegetable beverage. These components may be used independently or in combination. In this case, although the proportion of the additive is not very important, it is generally selected in the range of 0.01 to about 20 parts by weight per 100 parts by weight of the component of the present invention.

In addition, in order to achieve the object of the present invention, the present invention provides a method for screening SARS-coronavirus-2 inhibitory drug, comprising:

the step of administering the drug to be screened to Vero cells;

the step of infecting SARS-coronavirus-2;

the step of measuring cell death by a microscope or MTS method;

the step of reacting with an antibody specific for double-stranded RNA; and

the step of treating of at least one compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperidenas a control;

In this case, in the case of cells not infected with the virus, the cell viability is regarded as 100%, and the viability of the infected cells is measured, but is not limited thereto.

In addition, in order to measure the dose-response of the drug, a method of measuring double-stranded RNA generated in the host when a virus is infected is used, and in this case, double-stranded RNA is detected using an antibody, but is limited thereto.

In the screening of the drug, the effect of the drug is compared by using the drug Remdesivir as a control, but any drug known as SARS-coronavirus-2 inhibitory drug in this field can be used, but is not limited thereto

Cells are treated with a drug diluted 5 times from 100 μM, the virus is simultaneously infected, and the cells are fixed 24 hours after infection and stained with an antibody, and the cells can be imaged with Operetta (PerkinElmer, Operetta) equipment. In this case, Harmony software can calculate the infection rate. The case of infection with only the virus is regarded as 100%, and the case of no infection is regarded as 0%, and the infection rate when the drug is treated is calculated. The concentration (EC₅₀) that inhibits viral infection by 50% can be calculated through Prism software (GraphPad, Prism). The concentration that reduces 50% cell viability (OC₅₀) is calculated as the cytotoxic concentration, and Selectivity Index (SI) is an index indicating how superior the compound is to toxicity, and can be calculated by dividing the OC₅₀ value by the EC₅₀ value, but various methods for evaluating the efficacy of a drug can be used

Advantages and features of the present invention, and methods of achieving them, will become apparent with reference to the embodiments described below in detail. However, the present invention is not limited to the embodiments disclosed below, but will be implemented in various different forms, and only the present embodiments are provided so that the disclosure of the present invention is complete, and to completely inform those of ordinary skill in the art to which the present invention belongs, the scope of the invention, and the invention is only defined by the scope of the claims.

### EXAMPLES

### Example 1: Analysis Method

\textcircled{1} Seeding (2×10⁴ cells/well) of Vero cells in a 96 well plate and incubating them uniformly.

\textcircled{2} On the day of the experiment, remove the culture solution and add a new culture solution with various compounds diluted to an appropriate concentration (generally from 100 uM to 3-fold dilution).

\textcircled{3} Infect the culture medium to which the compound is added with SARS-CoV-2.

\textcircled{4} In the presence of the drug, the virus-infected cells are cultured at an appropriate temperature for 2-3 days.

\textcircled{5} After removing the culture medium to measure cell viability, it can be treated with MTS solution to measure absorbance at 490 nm or to obtain an image of cells with an Operetta (PerkinElmer, Operetta) device.

\textcircled{6} Set the cell control to 100% and the virus control to 0% to calculate the cell viability for each compound as the drug efficacy value (EC₅₀).

\textcircled{7} Calculate the concentration that reduces cell viability by 50% (CC₅₀) as the cytotoxic concentration.

### Example 2: Structure and Data of the Compound

1. Almitrine Bismesylate

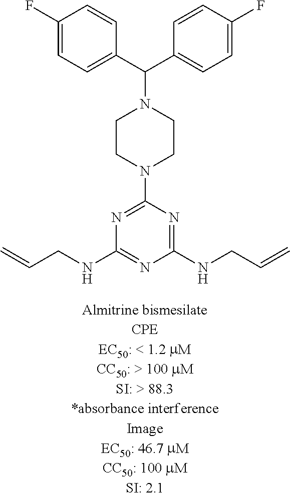 [SMILES: C=CCNc1nc(NCC=C)nc(N2CCN(C(c3ccc(F)cc3)c3ccc(F)cc3)CC2)n1]

2. Hydroxyzine Dihydrochloride

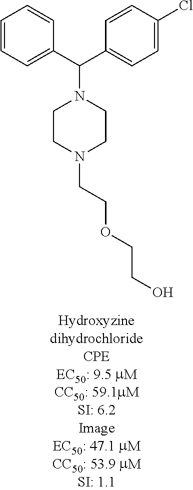 [SMILES: OCCOCCN1CCN(C(c2ccccc2)c2ccc(Cl)cc2)CC1]

3. Chlorcyclizine Hydrochloride

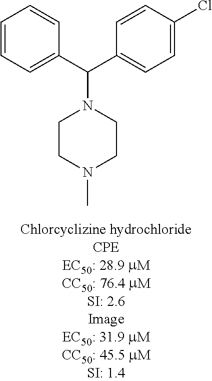 [SMILES: CN1CCN(C(c2ccccc2)c2ccc(Cl)cc2)CC1]

4. Cinnarizine

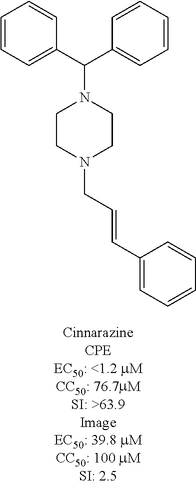 [SMILES: C(=C\c1ccccc1)/CN1CCN(C(c2ccccc2)c2ccccc2)CC1]

5. Cyclizine

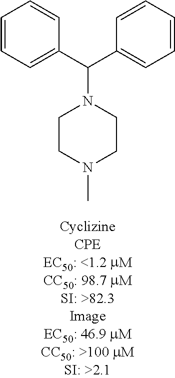 [SMILES: CN1CCN(C(c2ccccc2)c2ccccc2)CC1]

6. Olphenadrine Citrate

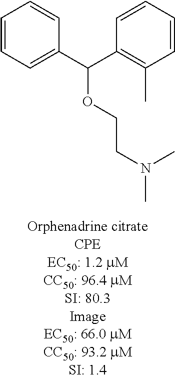 [SMILES: Cc1ccccc1C(OCCN(C)C)c1ccccc1]

7. Brompheniramine Maleate

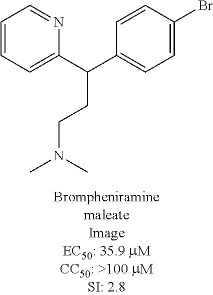 [SMILES: CN(C)CCC(c1ccc(Br)cc1)c1ccccn1]

8. Tolterodine Tartrate

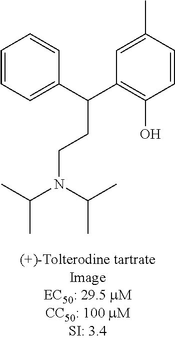 [SMILES: Cc1ccc(O)c(C(CCN(C(C)C)C(C)C)c2ccccc2)c1]

9. Cibenzoline Succinate

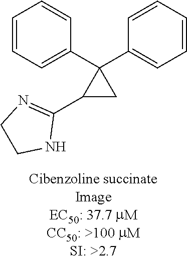 [SMILES: c1ccc(C2(c3ccccc3)CC2C2=NCCN2)cc1]

10. Zimellidine Dihydrochloride Monohydrate

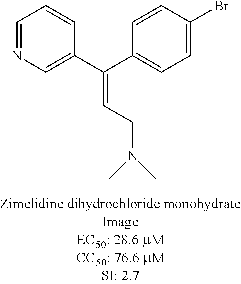 [SMILES: CN(C)C/C=C(\c1ccc(Br)cc1)c1cccnc1]

11. Diphenoxylate Hydrochloride

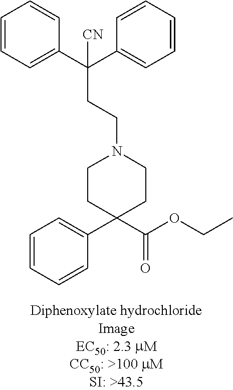 [SMILES: CCOC(=O)C1(c2ccccc2)CCN(CCC(C#N)(c2ccccc2)c2ccccc2)CC1]

12. Pipenzolate Bromide

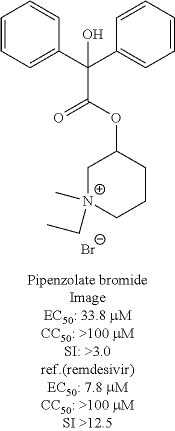 [SMILES: CC[N+]1(C)CCCC(OC(=O)C(O)(c2ccccc2)c2ccccc2)C1 ; [Br-]]

13. Predinol Methanesulfonate

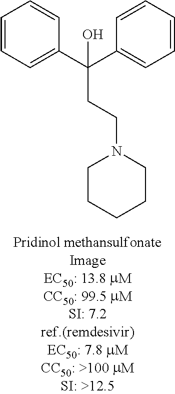 [SMILES: OC(CCN1CCCCC1)(c1ccccc1)c1ccccc1]

14. Procyclidine Hydrochloride

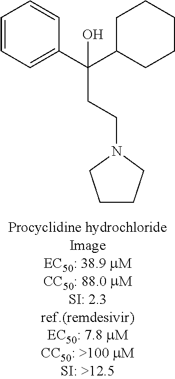 [SMILES: OC(CCN1CCCC1)(c1ccccc1)C1CCCCC1]

15. Fexofenadine Hydrochloride

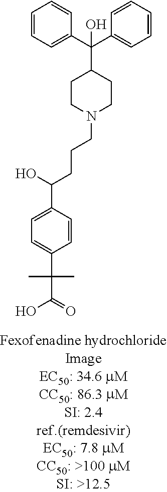 [SMILES: CC(C)(C(=O)O)c1ccc(C(O)CCCN2CCC(C(O)(c3ccccc3)c3ccccc3)CC2)cc1]

16. Biperiden

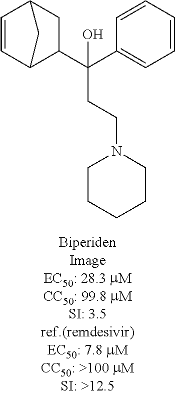 [SMILES: OC(CCN1CCCCC1)(c1ccccc1)C1CC2C=CC1C2]

1. A method of preventing or treating a viral infection comprising administering to a subject in need thereof an antiviral composition comprising at least one compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperiden.

2. The method of claim 1, wherein the virus is a coronavirus.

3. The method antiviral composition of claim 2, wherein the coronavirus is SARS-coronavirus-2, SARS or MERS.

4. The method of claim 1, wherein the composition is a pharmaceutical composition for preventing or treating a viral infection comprising—a pharmaceutically acceptable carrier

5. The method of claim 4, wherein the virus is a coronavirus.

6. The method of claim 5, wherein the coronavirus is SARS-coronavirus-2, SARS or MERS.

7. The method of claim 4, wherein the viral infection comprises flu, cold, sore throat, bronchitis, or pneumonia.

8. The method of claim 1, wherein the composition is a food composition for preventing or improving viral infection.

9. The method of claim 8, wherein the virus is a coronavirus.

10. The method of claim 9, wherein the coronavirus is SARS-coronavirus-2, SARS or MERS.

11. The method of claim 8, wherein the viral infection comprises flu, cold, sore throat, bronchitis, or pneumonia.

12. A method for screening a SARS-coronavirus-2 inhibitory drug, comprising:
administering a drug to be screened to Vero cells;
infecting with SARS-coronavirus-2;
measuring cell death by a microscope or MTS method;
reacting with an antibody specific for double-stranded RNA; and
treating as a control with at least a compound selected from the group consisting of Almitrine bismesylate, Hydroxyzine dihydrochloride, Chlorcyclizine hydrochloride, Cinnarizine, Cyclizine, Orphenadrine citrate, Brompheniramine maleate, Tolterodine tartrate, Cibenzoline succinate, Zimelidine dihydrochloride monohydrate, Diphenoxylate hydrochloride, Pipenzolate bromide, Pridinol methansulfonate, Procyclidine hydrochloride, Fexofenadine hydrochloride and Biperidenas.
